SEQUENCE LISTING (8 sequences)

SEQ ID NO: 1 - protein, 21 aa, synthetic construct
YSCCPEAYPDRVVSDGEVLYM

SEQ ID NO: 2 - protein, 22 aa, synthetic construct
PSHSSDWALTRDSWTTRQWLYM

SEQ ID NO: 3 - protein, 31 aa, synthetic construct
PSHSSYSCCPEAYPDRVVSDGEVLYMTTRQW

SEQ ID NO: 4 - protein, 34 aa, synthetic construct
WQRTTWYSCCPEAYPDRVVSDGEVLYMSDRTLAW

SEQ ID NO: 5 - protein, 12 aa, synthetic construct
RNRHTHLRTRPR

SEQ ID NO: 6 - protein, 9 aa, synthetic construct
YSCCPEAYP

SEQ ID NO: 7 - protein, 12 aa, synthetic construct
DRVVSDGEVLYM

SEQ ID NO: 8 - protein, 13 aa, synthetic construct
WQRTTWSDRTLAW

(19) United States
(12) Patent Application Publication
(10) Pub. No.: US 2026/0168991 A1
(43) Pub. Date: Jun. 18, 2026

(54) METHOD FOR DETECTION OF PESTICIDES USING PEPTIDE SEQUENCES

(71) Applicant: Imam Abdulrahman Bin Faisal University, Dammam (SA)

(72) Inventor: Sarah ALDULAIJAN, Dammam (SA)

(73) Assignee: Imam Abdulrahman Bin Faisal University, Dammam (SA)

(21) Appl. No.: 19/049,127

(22) Filed: Feb. 10, 2025

Related U.S. Application Data
(60) Provisional application No. 63/733,268, filed on Dec. 12, 2024.

(51) Int. Cl. G01N 33/53

(52) U.S. Cl. CPC G01N 33/5308; G01N 2333/001

(57) ABSTRACT

Aspects of the present disclosure are directed to a method for detection of pesticides using peptides. The method includes contacting at least one peptide, selected from a group consisting of YSM21, PSM22, PSW31, and WQA34, with a sample comprising a pesticide. The peptide comprises a fluorescent probe and the pesticide is imidacloprid. The method further includes binding the pesticide to the peptide to form an analyte composition and detecting the pesticide based on a fluorescence spectrum of the analyte composition.

### CROSS REFERENCE TO RELATED APPLICATION

This application claims the benefit of U.S. Provisional Application No. 63/733,268, filed Dec. 12, 2024, which is incorporated herein by reference in its entirety.

### STATEMENT REGARDING PRIOR DISCLOSURE BY THE INVENTORS

Aspects of the present disclosure are described in Aldulaijan, S., “In-silico selection of peptides for the recognition of imidacloprid” published in Volume 18, Issue 12, PLOS ONE, which is incorporated herein by reference in its entirety.

### REFERENCE TO SEQUENCE LISTING

In accordance with 37 CFR § 1.52 (e) (5) and with 37 CFR § 1.831, the specification makes reference to a Sequence Listing submitted electronically as a .xml file named 556116US Sequence Listing. The .xml file was generated on Feb. 13, 2025, and is 8,127 bytes in size. The entire contents of the Sequence Listing are hereby incorporated by reference.

### BACKGROUND

### Technical Field

The present disclosure is directed to a method for detection of pesticides, and more particularly, a method for detection of pesticides based on peptides having specific binding sites for the pesticides.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Imidacloprid, also known as 1-(6-chloro-3-pyridylmethyl)-2-nitroimidazolidine, is a frequently used insecticide that belongs to the class of neonicotinoids and mimics the structure of nicotine. It is a systemic insecticide and acts as an agonist of nicotinic acetylcholine receptors (nAChRs). It acts on the nervous system of the insects leading to their paralysis and death; however, the use of imidacloprid has raised environmental concerns, particularly regarding its effects on non-target organisms including mammals. One adverse effect of imidacloprid in mammals is that nAChRs may dysregulate endogenous ACh-elevated catecholamine synthesis in chromaffin cells of mammalian adrenal glands, ultimately increasing the adrenaline release from this endocrine organ [Kawahata, I. and Yamakuni, T., Imidacloprid, a neonicotinoid insecticide, facilitates tyrosine hydroxylase transcription and phenylethanolamine N-methyltransferase mRNA expression to enhance catecholamine synthesis and its nicotine-evoked elevation in PC12D cells Toxicology, 2018, 394, 84-92].

Environmental monitoring and food safety analysis of pesticides have devised various methods for the detection of such pesticides, including chromatography, immunoassays (such as enzyme-linked immunosorbent assays (ELISA)), and electrochemical methods. Chromatography based methods, including high-performance liquid chromatography (HPLC), separate insecticides from other substances based on the insecticides' interactions with the column's stationary phase, which can then be detected using specific detectors. Gas chromatography mass spectrometry (GC-MS) may be used when the insecticide is present in a complex matter such as soil. The sample may be vaporized and the components of the sample may be separated in the gas chromatograph. The mass spectrometer then identifies each component by its mass-to-charge ratio. These methods are specific but less commonly used as they need expensive equipment and trained personnels making them less accessible for routine or field applications.

Immunoassays, including ELISA, provide quick results and are more user friendly. For example, antibodies specific for insecticides, such as imidacloprid, can be employed in ELISA followed by colorimetric detection that can be quantified. Immunoassays have an advantage of being cost effective and allowing large numbers of samples to be screened at a fast rate; however, these assays are less sensitive and specific when compared to chromatographic methods, and the antibodies may sometimes cross react with compounds other than the target compound giving false positives and negatives.

Insecticides may also be detected using electrochemical methods where the electroactive properties of the pesticide are utilized in detection. Various techniques, such as cyclic voltammetry and differential pulse voltammetry, can be used to measure current response of a pesticide as the potential is varied to indicate the presence of the insecticide. Electrochemical methods are less complicated and cost effective, having the potential for real-time and on-site analysis; however, their sensitivity can be affected by the presence of other electroactive substances, and they may require calibration against known standards to ensure accurate measurements.

Biosensors are currently used in different fields of applications, such as diagnostics, food safety, and environmental monitoring, because of their cost-effectiveness and amiability [Baachaoui, S. et al., Laser-induced porous graphene electrodes from polyketimine membranes for paracetamol sensing, Royal Society Open Science, 2023, 10, 230294; Meftah, M. et al., Sensitive electrochemical detection of polymorphisms in IL6 and TGF beta 1 genes from ovarian cancer DNA patients using EcoRI and DNA hairpin-modified gold electrodes, Microchim Acta, 2023, 190, 1, 15; Baachaoui, S. et al., A Magnetoelectrochemical Bioassay for Highly Sensitive Sensing of Point Mutations in Interleukin-6 Gene Using TMB as a Hybridization Intercalation Indicator, Biosensors (Basel), 2023, 13, 2; Ouedraogo, B. et al., Laser-induced graphene electrodes on polyimide membranes modified with gold nanoparticles for the simultaneous detection of dopamine and uric acid in human serum, Microchim Acta, 2023, 190, 8, 316; Algethami, F. K. et al., Highly sensitive capacitance-based nitrite sensing using polydopamine/AuNPs-modified screen-printed carbon electrode, RSC Adv., 2023, 13, 31, 21336-44; and Algethami, F. K. et al., In silico selection of an aptamer for the design of aptamer-modified magnetic beads bearing ferrocene co-immobilized label for capacitive detection of acetamiprid, Talanta, 2023, 258]. Peptides have been examined for their uses as recognition elements for various compounds and molecules [Mastouri, M. et al, In silico screening for oligopeptides useful as capture and reporting probes for interleukin-6 biosensing, RSC Adv., 2022, 12, 21, 13003-13; Pavan, S. and Berti, F., Short peptides as biosensor transducers, Anal Bioanal Chem, 2012, 402, 10, 3055-70; Barbosa, A. J. M. et al., Protein- and Peptide-Based Biosensors in Artificial Olfaction, Trends Biotechnol., 2018, 36, 12, 1244-58; Puiu, M. and Bala, C., Peptide-based biosensors: From self-assembled interfaces to molecular probes in electrochemical assays, Bioelectrochemistry, 2018, 120, 66-75; Liu, Q. et al., Peptide-based biosensors, Talanta, 2015, 136, 114-27; and Erak, M. et al., Peptide chemistry toolbox—Transforming natural peptides into peptide therapeutics, Bioorg Med Chem., 2018, 26, 10, 2759-65]. Peptides are stable, cost effective, and easy to prepare through standard protocols. Peptides can also be modified to fit specific applications [Mastouri, M. et al., In silico screening for oligopeptides useful as capture and reporting probes for interleukin-6 biosensing, RSC Adv., 2022, 12, 21, 13003-13; Erak, M. et al., Peptide chemistry toolbox—Transforming natural peptides into peptide therapeutics, Bioorg Med Chem, 2018, 26, 10, 2759-65; Zambrano-Mila, M. S. et al., Peptide Phage Display: Molecular Principles and Biomedical Applications, Ther Innov Regul Sci, 2020, 54, 2, 308-17; and MacCulloch, T. et al., Emerging applications of peptide-oligonucleotide conjugates: bioactive scaffolds, self-assembling systems, and hybrid nanomaterials, Org Biomol Chem, 2019, 17, 7, 1668-82]. Peptides are suitable for detecting proteins [Erak, M. et al., Peptide chemistry toolbox—Transforming natural peptides into peptide therapeutics, Bioorg Med Chem, 2018, 26, 10, 2759-65; and Arya, S. K. et al., Label free biosensor for sensitive human influenza virus hemagglutinin specific antibody detection using coiled-coil peptide modified microelectrode array based platform, Sensors and Actuators B: Chemical, 2014, 194, 127-33], nucleic acids [O'Neil, K. T. et al., Design of DNA-binding peptides based on the leucine zipper motif, Science, 1990, 249, 4970, 774-8], bacteria [Pardoux, E. et al., Antimicrobial Peptides as Probes in Biosensors Detecting Whole Bacteria: A Review, Molecules, 2020, 25, 8], and chemicals compounds [Barbosa, A. J. M. et al., Protein- and Peptide-Based Biosensors in Artificial Olfaction, Trends Biotechnol, 2018, 36, 12, 1244-58; Erak, M. et al., Peptide chemistry toolbox—Transforming natural peptides into peptide therapeutics, Bioorg Med Chem, 2018, 26, 10, 2759-65; and Ding, X. et al., Oligopeptides functionalized surface plasmon resonance biosensors for detecting thiacloprid and imidacloprid, Biosens Bioelectron, 2012, 35, 1, 271-6]. Peptides can be easily obtained by chemical synthesis methods and avoid the need for in-depth and laborious procedures, such as in the case of antibodies [Sfragano, P. S. et al., The Role of Peptides in the Design of Electrochemical Biosensors for Clinical Diagnostics, Biosensors (Basel), 2021, 11, 8]. Peptide-based biosensor applications are increasing, and compounds and methods based on peptides for detection of pesticides are being developed.

CN110627872A describes a polypeptide with antibody specific for imidacloprid. The polypeptide detects imidacloprid in environmental and agricultural applications; however, CN110627872A describes a phage display technique and an ELISA method for the detection of imidacloprid.

CN106950383A describes applications of spider acetylcholine binding protein in ligand screening of a ligand gated ion channel. The ligand of the protein includes naturally occurring ligands, such as acetylcholine, gamma aminobutyric acid, glycine and serotonin and non-naturally occurring ligands, such as artificially synthesized imidacloprid.

There remains a need to develop methods for detection of pesticides which are cost effective, user friendly, and provide rapid results. Accordingly, an object of the present disclosure to provide a method for the detection of pesticides which is sensitive and selective for a target to overcome drawbacks of the current art. Another object of the present disclosure is to provide a method for pesticide detection based on peptides that is simple to operate and promotes accurate results.

### SUMMARY

In an exemplary embodiment, a method of pesticide detection is described. The method includes contacting at least one peptide with a sample comprising a pesticide, wherein the peptide is selected from a group consisting of YSM21, PSM22, PSW31, and WQA34. The YSM21 peptide has an amino acid sequence at least 85% identical to SEQ ID No.: 1. The PSM22 peptide has an amino acid sequence at least 85% identical to SEQ ID No.: 2. The PSW31 peptide has an amino acid sequence at least 85% identical to SEQ ID No.: 3. The WQA34 peptide has an amino acid sequence at least 85% identical to SEQ ID No.: 4. The peptide comprises a fluorescent probe, and the pesticide is imidacloprid. The method further includes binding the pesticide to the peptide to form an analyte composition and detecting the pesticide based on a fluorescence spectrum of the analyte composition.

In some embodiments, the peptide has a binding affinity for imidacloprid of −3 to −6 kcal/mol.

In some embodiments, the peptide is WQA34, and a hydroxyl group of the imidacloprid interacts with a tryptophan in a sixth position of the WQA34 during the binding.

In some embodiments, the peptide is WQA34, and a benzene ring of the imidacloprid interacts with a tryptophan in a thirty fourth position of the WQA34 during the binding.

In some embodiments, the peptide is WQA34, and a chloride atom of the imidacloprid interacts with a tryptophan in a thirty fourth position of the WQA34 and a proline in a fifteenth position of the WQA34 during the binding.

In some embodiments, the peptide is WQA34, and the peptide has an average binding free energy of −7 to −6 kcal/mol to the imidacloprid during the binding.

In some embodiments, the peptide is WQA34, and the peptide has an average free energy of solvated receptor-ligand binding of −6 to −4 kcal/mol to the imidacloprid during the binding.

In some embodiments, the peptide is WQA34, and the peptide has an equilibrium dissociation constant of 30 to 50 μM to the imidacloprid during the binding.

In some embodiments, the peptide is WQA34, and a root mean square deviation value of the peptide associated with the imidacloprid during the binding is 70 to 80% less compared to a root mean square value of the peptide not associated with the imidacloprid during the binding.

In some embodiments, the peptide is PSW31, and a root mean square deviation value of the peptide associated with the imidacloprid during the binding is 45 to 55% less compared to a root mean square value of the peptide not associated with the imidacloprid during the binding.

In some embodiments, the method further includes binding the peptide to acetamiprid and clothianidin.

In some embodiments, the peptide is WQA34, and the peptide has an average free energy of −3 to −1 kcal/mol for acetamiprid during the binding.

In some embodiments, the peptide is WQA34, and the peptide has an average free energy of −2 to 0 kcal/mol for clothianidin during the binding.

In some embodiments, the peptide is YSM21 and has an amino acid sequence at least 95% identical to SEQ ID No.: 1.

In some embodiments, the peptide is PSM22 and has an amino acid sequence at least 95% identical to SEQ ID No.: 2.

In some embodiments, the peptide is PSW31 and has an amino acid sequence at least 95% identical to SEQ ID No.: 3.

In some embodiments, the peptide is WQA34 and has an amino acid sequence at least 90% identical to SEQ ID No.: 4.

In some embodiments, the peptide is WQA34 and has an amino acid sequence at least 95% identical to SEQ ID No.: 4.

In some embodiments, the peptide is WQA34 and has an amino acid sequence at least 99% identical to SEQ ID No.: 4.

In some embodiments, the peptide is PSW31, and the peptide has an equilibrium dissociation constant of 30,000 to 40,000 μM to the imidacloprid during binding.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a flow chart of a method of detection of pesticides, according to certain embodiments.

FIG. 2 depicts 3D structures for imidacloprid (IMI) and four peptides: YSM21, PSM22, PSW31, and WQA34, according to certain embodiments.

FIG. 3A depicts molecular docking interactions between IMI and YSM21, according to certain embodiments.

FIG. 3B depicts molecular docking interactions between IMI and PSM22, according to certain embodiments.

FIG. 3C depicts molecular docking interactions between IMI and PSW31, according to certain embodiments.

FIG. 3D depicts molecular docking interactions between IMI and WQA34, according to certain embodiments.

FIG. 4A depicts root mean square deviation (RMSD) for free and associated peptides, according to certain embodiments.

FIG. 4B depicts root mean square deviation (RMSD) for peptides vs. time, according to certain embodiments.

FIG. 5A depicts the chemical structure of three neonicotinoid pesticides, according to certain embodiments.

FIG. 5B depicts the plots of root mean square deviation (RMSD) and the average free energy determined for the selectivity of WQA34 peptide, according to certain embodiments.

FIG. 6 depicts the structure of the Lymnaea stagnalis acetylcholine-binding protein Q55R mutant complex (PDB ID 3WTH), according to certain embodiments.

FIG. 7A depicts 3D structures of the position of IMI between chain A and chain B, according to certain embodiments.

FIG. 7B depicts 3D structures of interactions between IMI and the chain A and B residues, according to certain embodiments.

FIG. 8A depicts the 3D structure of RNR12 peptide, according to certain embodiments.

FIG. 8B depicts the 3D structure of interactions between IMI and RNR12 ligand from molecular docking calculations, according to certain embodiments.

FIG. 9A depicts the number of contacts for the peptide vs. time, according to certain embodiments.

FIG. 9B depicts the minimum number of contacts for each peptide, according to certain embodiments.

### DETAILED DESCRIPTION

When describing the present disclosure, the terms used are to be construed in accordance with the following definitions, unless a context dictates otherwise.

Embodiments of the present disclosure will now be described more fully hereinafter with reference to the accompanying drawings wherever applicable, in that some, but not all embodiments of the disclosure are shown.

In the drawings, like reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “peptide” refers to a sequence of amino acids, linked by peptide bonds (an amide type of covalent chemical bond linking two consecutive alpha-amino acids from C1 (carbon number one) of a first alpha-amino acid and N2 (nitrogen number two) of a second alpha-amino acid), of a certain length.

As used herein, the term “receptor” refers to a protein present on the surface of or within a cell that binds to specific molecules to trigger a response related to cellular processes. Receptors are chemical structures that receive and transduce signals that may be integrated into biological systems.

As used herein, the term “ligand” refers to an ion or a molecule with a functional group that binds to another molecule to form a complex that can initiate a biological effect.

As used herein, the term “pesticide” refers to a chemical substance and/or a biological agent designed to target specific biological pathways or functions within a pest, leading to its death or inhibition.

As used herein, the term “insecticide” refers to a category of pesticide that targets insects, i.e., pests such as mosquitoes, ants, weevils, grasshoppers, beetles, and caterpillars.

As used herein, the term “neonicotinoids” refers to a class of insecticides that are chemically similar in structure to nicotine.

As used herein, the term “binding specificity” refers to the ability of a molecule to selectively interact with a particular target molecule and/or set of molecules. Binding specificity of a peptide refers to the ability of the peptide to selectively interact with a particular molecule, the ability being the result of a specific arrangement of amino acids in the peptide sequence.

As used herein, the term “analyte” refers to a substance and/or chemical component that is of interest and is being measured or analyzed in a sample in analytical procedures.

As used herein, the term “probe” refers to specific chemical and/or biological molecules designed to detect the presence and/or measure the quantities of another molecule in a composition.

The present disclosure is intended to include all hydration states of a given compound or formula, unless otherwise noted or when heating a material.

Aspects of the present disclosure are directed to a method of detection for pesticides using peptides. More particularly, aspects of the present disclosure are directed to a method for detection of imidacloprid using low-cost receptors designed from peptides selective for imidacloprid. Three primary short peptides (YSP09, DMR12, and WQW13 having 9, 12, and 13 amino acids (AA) in length, respectively) from the pesticide interacting zones with A, B, and C chains of the nicotinic receptor from Lymnaea stagnalis acetylcholine-binding protein Q55R mutant receptor-imidacloprid complex were selected. Using molecular docking and molecular dynamics (MD) simulations, the three peptides were observed to form complexes with the target, imidacloprid. Combination of these peptides allowed preparing a set of longer peptides (YSM21, PSM22, PSW31, and WQA34) that have higher stability and binding affinity. In particular, the WQA34 peptide displayed an average binding free energy of −6.44±0.27 kcal/mol, which is three times higher than that of a reference RNR12 peptide (−2.29±0.25 kcal/mol) and formed a stable complex with imidacloprid. Dissociation constants (K_{d}), calculated from the binding free energy display that WQA32 (40 μM) has a K_{d} value three orders of magnitude lower than the reference RNR12 peptide (3.4× 10⁴ μM).

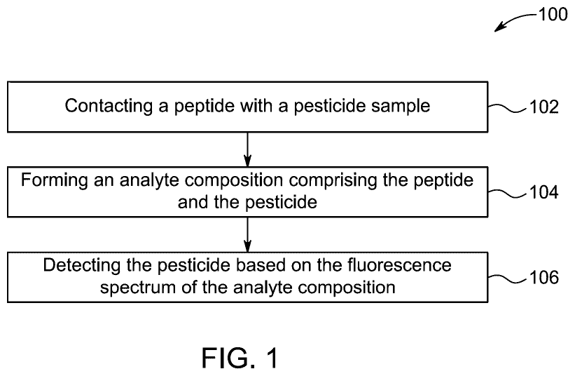

FIG. 1 illustrates a schematic flow chart of a method 100 of detection of pesticides. The order in which the method 100 is described is not intended to be construed as a limitation, and any number of the described method steps can be combined in any order to implement the method 100. Additionally, individual steps may be removed or skipped from the method 100 without departing from the spirit and scope of the present disclosure.

At step 102, the method 100 includes contacting a peptide with a sample comprising a pesticide. The pesticides may be synthetic, organic, and/or inorganic. In one embodiment, the pesticides may be commonly used pesticides. In a specific embodiment, the pesticides belong to a group comprising organophosphates, rotenones, carbamates, pyrethroids, neonicotinoids, and the like. In a preferred embodiment, the pesticides may belong to a group of neonicotinoids. In embodiments where the pesticide belongs to neonicotinoids group, the pesticide may be imidacloprid, acetamiprid, clothianidin, thiamethoxam, and dinotefuran. In one embodiment, the pesticide is acetamiprid. In another embodiment, the pesticide is clothianidin. In a preferred embodiment, the pesticide is imidacloprid. In some embodiments, the pesticide is imidacloprid, acetamiprid, and clothianidin.

The peptide of method 100 may be chosen based on its binding specificity for the pesticide. Specific binding regions of a pesticide may be present on the peptide chain and peptides having one or more binding regions for a particular pesticide may be selected. In an embodiment where the pesticide is an organophosphate or a carbamate, the peptide may have binding regions similar or identical to the sequence of acetylcholinesterase enzymes. In other embodiments where the pesticide is a neonicotinoid, the peptide may have binding regions similar or identical to the sequence of a nicotinic acetylcholine receptor (nAChRs) protein.

Peptides having amino acid sequences similar to mutant proteins in the host also form a part of the present disclosure. Accordingly, certain embodiments of the disclosure include peptides having sequences similar or identical to the mutant acetylcholine binding protein of the host. One or more of such peptide sequences may be selected. In some embodiments, the number of peptide sequences may vary from 1 to 10. In specific embodiments, short peptide sequences having about 9 to 13 amino acids may be synthesized, which can be combined in a certain order to obtain larger peptide sequences having about 20 to 35 amino acids.

In an exemplary embodiment, the present disclosure includes a YSM21 peptide having an amino acid sequence at least 85% identical to SEQ ID No.: 1, a PSM22 peptide having an amino acid sequence at least 85% identical to SEQ ID No.: 2, a PSW31 peptide having an amino acid sequence at least 85% identical to SEQ ID No.: 3, and a WQA34 peptide having an amino acid sequence at least 85% identical to SEQ ID No.: 4. In some embodiments, the present disclosure includes seven peptide sequences denoted as YSP09 (SEQ ID No.: 5), DRM12 (SEQ ID No.: 6), WQW13 (SEQ ID No.: 7), YSM21 (SEQ ID No.: 1), PSM22 (SEQ ID No.: 2), PSW31 (SEQ ID No.: 3), WQA34 (SEQ ID No.: 4), and a reference peptide sequence, RNR12 (SEQ ID No.: 8). Peptide sequences having SEQ ID Nos. 5 to 7 are derived from the A, B, and C chains of the acetylcholine binding protein mutant Q55R of Lymnaea stagnalis and the peptide sequences having SEQ ID Nos. 1 to 4 are derived by combining SEQ ID Nos. 5 to 7.

The exemplified peptide sequences may further include fragments, variants, and derivatives thereof having some or all the binding specificity for the pesticide. Variations in the peptide sequence may be obtained by substitutions, deletions, and/or additions of amino acids in the sequence, so long as at least 90% of the peptide sequence remains identical to the parent peptide sequence. Accordingly, in one embodiment, the sequence for peptide YSM21 is at least 95% identical to SEQ ID No.: 1. In another embodiment, the sequence for peptide PSM22 is at least 95% identical to SEQ ID No.: 2. In some embodiments, the sequence for peptide PSW31 is at least 95% identical to SEQ ID No.: 3. In some other embodiments, the sequence for peptide WQA34 is at least 90% identical to SEQ ID No.: 4, preferably at least 95% identical to SEQ ID No.: 4, and more preferably at least 99% identical to SEQ ID No.: 4.

At step 104, the method 100 includes forming an analyte composition comprising the peptide and the pesticide. The peptide binds to the pesticide via non-covalent interactions, preferably dispersion forces, dipole-dipole forces, and/or hydrogen bonding forces, at different sites. The binding affinity of the peptides for the pesticide depends on the number of interacting sites in the peptide sequence. Certain amino acids have stronger interactions with specific functional groups in the pesticide and form more stable complexes. In an embodiment, a tryptophan in a sixth position of the peptide WQA34 interacts with a hydroxyl group of the imidacloprid. In another embodiment, a tryptophan in a thirty fourth position of the peptide WQA34 interacts with a benzene ring of the imidacloprid. In yet another embodiment, a tryptophan in a thirty fourth position of the peptide WQA34 and a proline in a fifteenth position of the peptide WQA34 interact with a chloride atom of the imidacloprid. In certain embodiments, the peptides may have a binding affinity for imidacloprid in the range of −3 to −6 kcal/mol, preferably −3.5 to −5.5 kcal/mol, preferably −4 to −5 kcal/mol, preferably −4.3 to −4.7 kcal/mol.

At step 106, the method 100 includes detecting the pesticide based on a fluorescence spectrum of the analyte composition. The peptides may be conjugated to a probe which may be fluorescently labelled to detect the binding interactions between the peptides and the insecticide. Fluorescent dyes may be incorporated in the peptide sequence at a desired position and can be visualized using techniques such as fluorescence microscopy. Suitable examples of fluorescent dyes that can be used to label peptides include, but are not limited to, fluorescein isothiocyanate (FITC), green fluorescent protein (GFP), iFluor, alexa flour, rhodamine dyes, Texas red, combinations thereof, and the like. Alternatively, certain amino acids that are intrinsically fluorescent may be inserted into the peptide sequences at desired positions. Such amino acids may emit light when excited by specific wavelengths. Fluorescent amino acids may include naturally occurring amino acids, like tryptophan and tyrosine, and non-natural amino acids, such as cyanotryptophan, acridonylalanine, and the like. In some embodiments, other methods of detection, such as ultraviolet-visible (UV-Vis) spectroscopy, refractive index, fluorescence, electrochemical, evaporative light scattering, precipitation, a combination thereof, and the like, may be used to detect the pesticide after binding the pesticide to the peptide.

Binding interactions between peptides and a pesticide result in the release of binding free energies. The value of average binding free energy for each peptide is an indicator of the strength of the peptide-pesticide complex. In an example where the pesticide is imidacloprid, the peptide WQA34 may have an average binding free energy of about-7 to −6 kcal/mol, preferably −6.9 to −6.1 kcal/mol, preferably −6.8 to −6.2 kcal/mol, preferably −6.7 to −6.3 kcal/mol, preferably −6.6 to −6.4 kcal/mol. The average free energy value of a solvated receptor-ligand complex is another parameter that determines the stability of receptor-ligand complexes. Higher free energy values indicate a more stable receptor-ligand complex. In cases where the ligand is imidacloprid, the peptide WQA34-imidacloprid complex exhibits an average free energy of about −6 to −4 kcal/mol. In some embodiments where the ligand is acetamiprid, the peptide WQA34 exhibits an average free energy of about −3 to −1 kcal/mol, preferably −2.5 to −1.5 kcal/mol, preferably −2.2 to −1.8 kcal/mol. In some embodiments, the peptide WQA34 exhibits an average free energy of about −2 to 0 kcal/mol, preferably −1.5 to −0.5 kcal/mol, preferably −1.2 to −0.8 kcal/mol, when the ligand is clothianidin. The exemplified peptides, particularly WQA34, show a higher value of average free energy when compared to a reference peptide, RNR12.

The stability of receptor-ligand complex can also be determined by measuring the values of dissociation constants for the peptides. The dissociation constants for the peptide complexes can be calculated from the average free binding energies of the complexes. In case of exemplified peptides-imidacloprid complexes, the dissociation constant values for YSM21, PSM22, and PSW31 peptide complexes range from 1.4×10⁴ to 3.4×10⁴, as they have lower average binding free energies and, hence, cannot form stable complexes. The peptide PSW31 has the highest dissociation constant value ranging from 3.4×10⁴ to 4×10⁴. In some embodiments, the dissociation constant value for the peptide WQA34 complex ranges from 30 to 50 M, preferably 35 to 45 μM, preferably 38 to 42 μM, which indicates the formation of a stable complex. In some embodiments, the dissociation constant value for the peptide WQA34 complex may be about 35vμM, about 40 μM, or about 45 μM.

Root mean square deviation (RMSD) values for peptides may be measured for determining the stability of peptide-imidacloprid complexes. RMSD values are inversely related to the stability of the complexes. Peptides associated with the ligand show a decrease in RMSD values when compared to their RMSD values in free states (i.e., no pesticide). In one embodiment, the RMSD value for the peptide WQA34 when associated with imidacloprid reduces by 70 to 80%, preferably 71 to 79%, preferably 72 to 78%, preferably 73 to 77%, preferably 74 to 76%, of its RMSD value when in free state. In another embodiment, the RMSD value for the peptide PSW31 when associated with imidacloprid is 45 to 55%, preferably 46 to 54%, preferably 47 to 53%, preferably 48 to 52%, preferably 49 to 51%, less than its RMSD value when in free state.

### Examples

The following examples describe and demonstrate a method for detection of pesticides and analysis of synthesized peptides as described herein. The examples are provided solely for illustration and are not to be construed as limitations of the present disclosure, as many variations thereof are possible without departing from the spirit and scope of the present disclosure.

### Example 1: Selection of Peptides

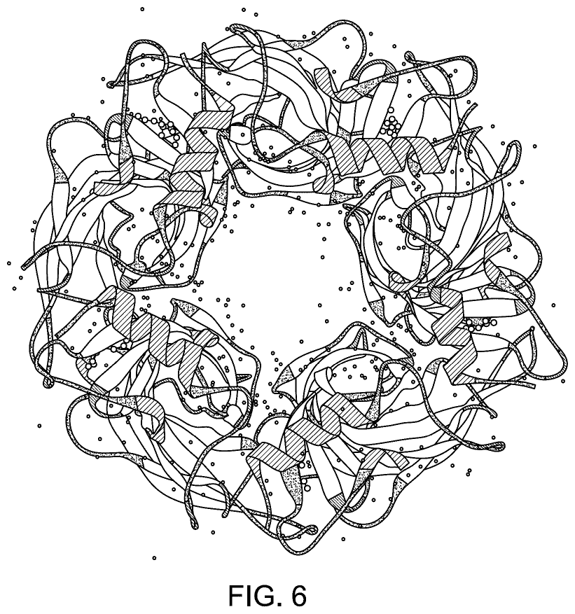

To identify concise peptides suitable for imidacloprid binding, investigation using structural insights derived from the Lymnaea stagnalis acetylcholine-binding protein Q55R mutant complex (PDB ID 3WTH) was initiated [Ihara, M. et al., Studies on an acetylcholine binding protein identify a basic residue in loop G on the β1 strand as a new structural determinant of neonicotinoid actions, Mol Pharmacol, 2014, 86, 6, 736-46, which is incorporated herein by reference in its entirety], accessible within the RCSB PDB database [Berman, H. M. et al., The Protein Data Bank, Nucleic Acids Res, 2000, 28, 1, 235-42, which is incorporated herein by reference in its entirety]. This pentameric protein represents a target for neonicotinoid insecticides in the insect kingdom. The architecture of each monomer is composed of a combination of beta sheets and helical structures interconnected by coil regions. The pesticides may stack Tyr185 with basic residues, such as Lys35 in loop 5 [Ihara, M. et al., Studies on an acetylcholine binding protein identify a basic residue in loop G on the β1 strand as a new structural determinant of neonicotinoid actions, Mol Pharmacol, 2014, 86, 6, 736-46, which is incorporated herein by reference in its entirety]. The starting protein is composed of five monomers in complex with five imidacloprid as shown in FIG. 6. Three peptides (SEQ. ID No.: 5, SEQ. ID No.: 6, and SEQ. ID No.: 7) were selected from the binding areas of the different monomer/imidacloprid complexes using the “PLIP” webserver software [Salentin, S. et al., PLIP: fully automated protein-ligand interaction profiler, Nucleic Acids Res, 2015, 43, W1, W443-7, which is incorporated herein by reference in its entirety], which was used to identify possible binding sites between the protein and the ligand. 3D forms of the peptides were retrieved using PEP-FOLD webserver software [Thevenet, P. et al., PEP-FOLD: an updated de novo structure prediction server for both linear and disulfide bonded cyclic peptides, Nucleic Acids Res, 2012, 40, W288-93; Lamiable, A. et al., PEP-FOLD3: faster de novo structure prediction for linear peptides in solution and in complex, Nucleic Acids Res, 2016, 44, W1, W449-54; and Shen, Y. et al., Improved PEP-FOLD Approach for Peptide and Miniprotein Structure Prediction, J Chem Theory Comput, 2014, 10, 10, 4745-58, which are incorporated herein by references in their entireties].

From the selection of peptides and the study of the different poses of interactions between the IMI and the three peptides, it was shown that longer peptides have more stable complexes and a higher affinity toward the ligand (IMI); therefore, several modifications and mutations were made to create four new peptides (SEQ. ID No.: 1, SEQ. ID No.: 2, SEQ. ID No.: 3, and SEQ. ID No.: 4) having lengths from 21 to 34 residues. PEP-FOLD was also used to generate the 3D structures of the peptides.

### Example 2: Molecular Docking

To evaluate the affinity between these peptides and imidacloprid, a molecular docking simulation using AutoDock VINA (v1.2.3) was performed [Trott, O. and Olson, A. J., AutoDock Vina: improving the speed and accuracy of docking with a new scoring function, efficient optimization, and multithreading, J Comput Chem, 2010, 31, 2, 455-61, which is incorporated herein by reference in its entirety], with the peptides serving as the receptors. To ensure reproducibility, the entire peptide was positioned within a defined grid box, which had dimensions of 66×40×40 Å³, and the hydrogens were added to polar atoms of the peptides. Imidacloprid was utilized as a ligand, and all bonds in the ligand were specified as rotatable, allowing for flexible conformational sampling throughout the docking process. These parameters and conditions were employed to substantiate the methods. AutoDock Tools (v1.5.7) [Morris, G. M. et al., AutoDock4 and AutoDockTools4: Automated docking with selective receptor flexibility, J Comput Chem, 2009, 30, 16, 2785-91, which is incorporated herein by reference in its entirety] was used to prepare input files. The pH of the systems is 7. PyMOL [The PyMOL Molecular Graphics System, NY, USA, Schrodinger, LLC, 2010, which is incorporated herein by reference in its entirety] and BIOVIA Discovery Studio Visualizer [BIOVIA DS, BIOVIA Discovery Studio Visualizer, v16.1.0.15350. San Diego, Dassault Systemes, 2015, which is incorporated herein by reference in its entirety] were used to analyze established interactions between peptides and ligand.

### Example 3: Molecular Dynamics (MD) Simulations

To look at the stability of the selected poses, MD simulations were carried out using NAMD software [Phillips J. C. et al., Scalable molecular dynamics on CPU and GPU architectures with NAMD, J Chem Phys, 2020, 153, 4, 044130, which is incorporated herein by reference in its entirety] for a duration of 480 nanoseconds (ns). Beforehand, the designed peptide-IMI complexes were minimized and equilibrated using NAMD software for 10,000 cycles. MD simulations using the complex structures were conducted with the CHARMM36 forcefield [Vanommeslaeghe, K. et al., CHARMM general force field: A force field for drug-like molecules compatible with the CHARMM all-atom additive biological force fields, J Comput Chem, 2010, 31, 4,671-90, which is incorporated herein by reference in its entirety]. The complexes were solvated in a cubic box with keeping 10 Å between the complex and the box edge. NaCl ions were added to neutralize the system charge. For all simulations, temperature was set at 310 K and the pressure was set to 1 bar to closely mimic the general wet-lab experimental conditions. Subsequently, full temperature and pressure equilibrated systems were used as the initial configurations for the MD production. All simulations were conducted using a 2 femtosecond (fs) time step.

Root mean square deviation (RMSD) values were calculated with tools included in VMD [Humphrey, W. et al., VMD: visual molecular dynamics, J Mol Graph, 1996, 14, 1, 33-8, 27-8, which is incorporated herein by reference in its entirety] to check the stability of the studied systems. MM-PBSA methods [Genheden, S. and Ryde, U., The MM/PBSA and MM/GBSA methods to estimate ligand-binding affinities, Expert Opin Drug Discov, 2015, 10, 5, 449-61, which is incorporated herein by reference in its entirety], implemented in the VMD CaFE plugin [Liu, H. and Hou, T., CaFE: a tool for binding affinity prediction using end-point free energy methods, Bioinformatics, 2016, 32, 14, 2216-8, which is incorporated herein by reference in its entirety] were used to calculate the average of free energy from 3 different trajectories between 440 ns and 480 ns simulation time, after convergence of the systems [Genheden, S. and Ryde, U., The MM/PBSA and MM/GBSA methods to estimate ligand-binding affinities, Expert Opin Drug Discov, 2015, 10, 5, 449-61; Wang, E. et al., End-Point Binding Free Energy Calculation with MM/PBSA and MM/GBSA: Strategies and Applications in Drug Design, Chemical Reviews, 2019, 119, 16, 9478-508; and Genheden, S. and Ryde, U., How to obtain statistically converged MM/GBSA results, J Comput Chem, 2010, 31, 4,837-46, which are incorporated herein by references in their entireties]. The binding free energy

$$( Δ {G}_{binding}^{slvd} )$$

can be decomposed into the relative free energy of the solvated recpetor-ligand complex

$$( Δ {G}_{sol}^{Cplx} )$$

and the separated solvated ligand

$$( Δ {G}_{sol}^{Lig} )$$

and receptor

$$( Δ {G}_{sol}^{Rec} ) ,$$

as shown in Eq 1:

$$Δ {G}_{binding}^{slvd} = Δ {G}_{sol}^{Cplx} - Δ {G}_{sol}^{Rec} - Δ {G}_{sol}^{Lig} \qquad Eq 1$$

Free energy (ΔG^{s}) can be further decomposed into four main contributions from the van der Waals (ΔE_{vdW}), the electrostatic interaction (ΔE_{elec}), polar solvation (ΔGₚₒₗ), and non-polar solvation (ΔGₙₒₙₚₒₗ), as shown in Eq 2:

$$Δ {G}^{s} = Δ {E}_{vdW} + Δ {E}_{elec} + Δ {G}_{pol} + Δ {G}_{nonpol} \qquad Eq 2$$

Dissociation constants were calculated from the values of binding free energies for the different peptide/IMI complexes [Paakkonen, J. et al., Calculation and Visualization of Binding Equilibria in Protein Studies, ACS Omega, 2022, 7, 12,10789-95, which is incorporated herein by reference in its entirety]. The dissociation constant (K_{d}) is linked to the free binding energy by the following equation:

$$Δ G = - RT ln {K}_{a} = RT ln {K}_{d} \qquad Eq 3$$

where R is the gas constant, T is the working temperature Kₐ and K_{d} are the association and the dissociation constants, respectively; however, a method of orthogonal distance regression to calculate the dissociation constant (K_{d}) was used and did not use the theoretical method mentioned in Eq 3.

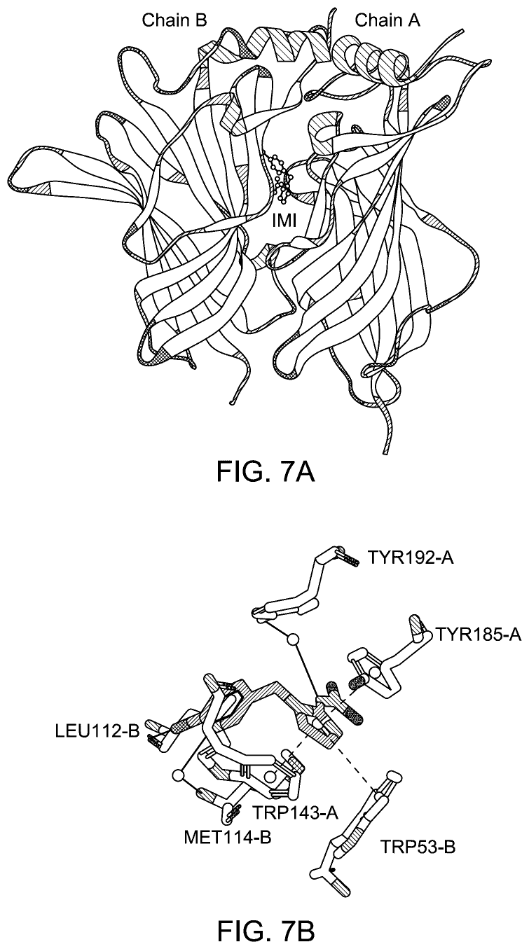

Analysis of the different complexes between the monomers and imidacloprid showed the existence of 7 amino acids (143A, 185A, 192A, 53B, 104B, 112B, and 114B from A and B chains) interacting with the target pesticide as shown in FIGS. 7A-7B. A sequence from A185 to A192 was selected to build part I (named YSP09 (SEQ. ID No.: 5)). Another sequence from 104B to 114B was selected to build part II (named DRM12 (SEQ. ID No.: 6)). A third peptide (named WQW13 (SEQ. ID No.: 7)), consisting of 13 amino acids located between TRP53 and TRP65 from chain C of the protein, was also selected since it interacts with imidacloprid, as supported by the PLIP analysis. These results indicate that the interaction between TRP53 and imidacloprid is facilitated by a x-stacking interaction between the imidacloprid molecule and the side chain of tryptophan residue at position 53 in the protein chain. Molecular docking shows that the 3 peptides have binding affinities ranging between −3.17 and −3.80 kcal/mol (entries 1 to 3 from Table 1).

TABLE 1
Sequences of the selected peptides, their codes, number of amino acids, and
binding affinity from docking

| Sequence | | | No. of | Binding |
|---|---|---|---|---|
| ID | Peptide | | amino | affinity |
| Number | Code^{a} | Sequences | acids | (kcal/mol) |
| 6 | YSP09 | YSCCPEAYP | 9 | −3.21 |
| 7 | DRM12 | DRVVSDGEVLYM | 12 | −3.17 |
| 8 | WQW13 | WQRTTWSDRTLAW | 13 | −3.80 |
| 1 | YSM21 | YSCCPEAYPDRVVSDGEVLYM | 21 | −4.30 |
| 2 | PSM22 | PSHSSDWALTRDSWTTRQWLYM | 22 | −4.61 |
| 3 | PSW31 | PSHSSYSCCPEAYPDRVVSDGEVLYMTTR | 31 | −4.80 |
| | | QW | | |
| 4 | WQA34 | WQRTTWYSCCPEAYPDRVVSDGEVLY | 34 | −5.30 |
| | | MSDRTLAW | | |
| 5 | RNR12^{b} | RNRHTHLRTRPR | 12 | −3.90 |

ªPeptide code is generated from the first two amino acids from the right followed by the last amino acid and total number of sequences
^{b}This peptide has previously been studied [Ding, X. et al., Oligopeptides functionalized surface plasmon resonance biosensors for detecting thiacloprid and imidacloprid, Biosens Bioelectron, 2012, 35, 1, 271-6, which is incorporated herein by reference in its entirety] and is run for sake of comparison

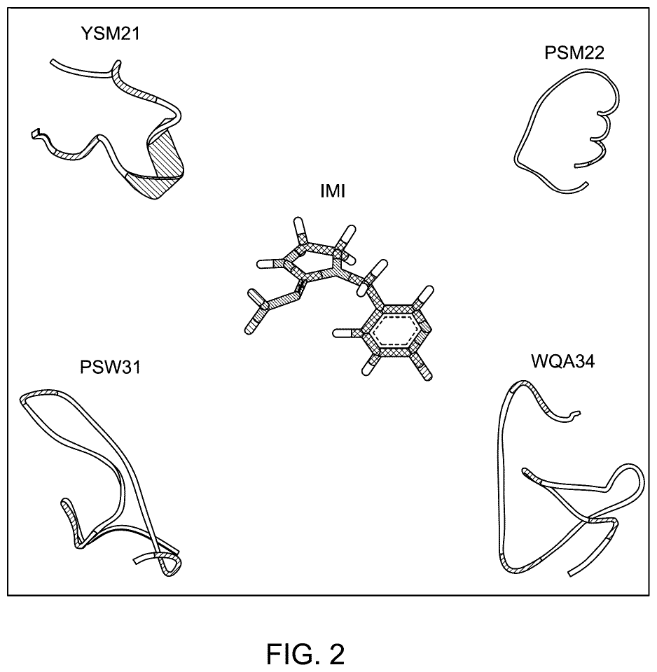

To improve interactions between the peptide sequences and IMI, three shorter peptides (entries 1 to 3 from Table 1) were combined to obtain four longer peptides (entries 4 to 7 from Table 1). YSP09 was linked with DRM12 from its N-terminal position to create a 21-amino acid sequence (YSM21, SEQ. ID No.: 1), which was tested for interactions with IMI. For a PSM22 peptide, an inverse order of WQW13 was an initial template, which was then modified by adding six (PSHSSD) and three (LYM) amino acids at C-terminal and N-terminal positions, respectively, to build a new peptide with a 22 amino acid sequence. PSW31 was built from YSM21, and the sequences were modified by adding the first five amino acids of PSM22 (PSHSS) at the C-terminal and the first five amino acids of WQW13 in reverse order (TTRQW) at the N-terminal position to form a sequence with 31 amino acids. The last peptide (WQA34) was obtained by adding the first six and the last seven amino acids from WQW13 to the C-terminal and N-terminal positions of peptide YSM21, respectively. 3D structures of the YSM21, PSM22, PSW31, WQW34, and IMI are shown in FIG. 2. Stability and affinity of the four combined peptides toward IMI was assessed using molecular docking.

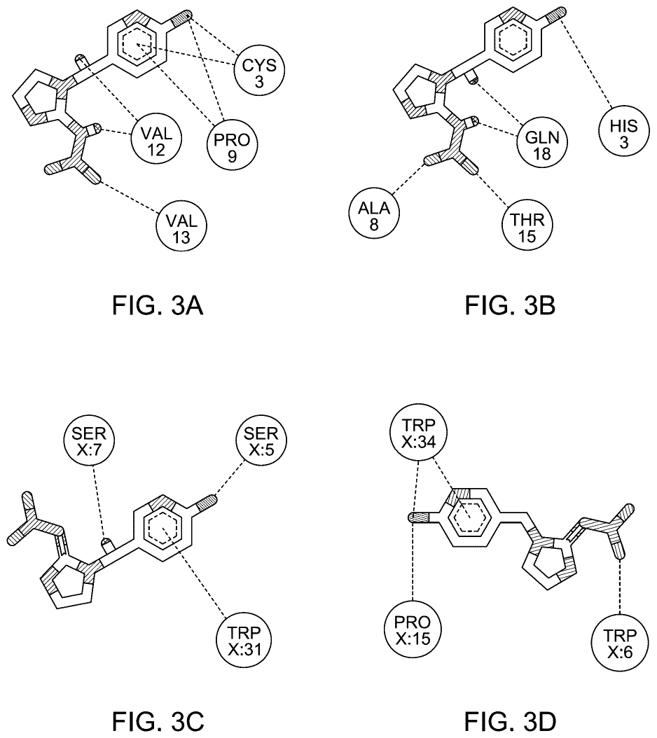

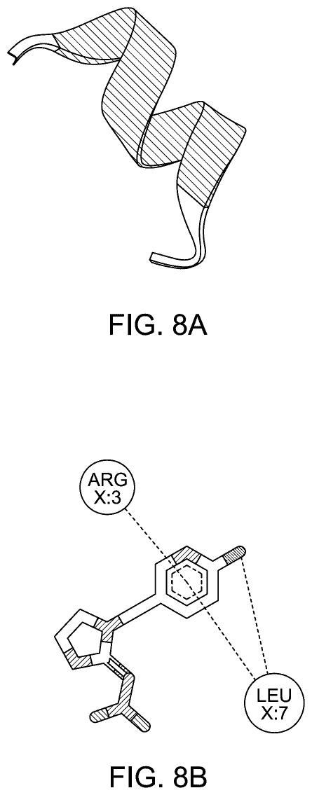

As shown in Table 1, YSM21 has the highest binding affinity value (−4.30 kcal/mol) of YSM21, PSM22, PSW31, and WQW34. FIGS. 3A-3D demonstrate imidacloprid interacting with YSM21, PSM22, PSW31, and WQW34, respectively. The YSM21 peptide establishes 3 hydrogen bonds (HBs) with IMI having a length of less than 4 Å. There is one conventional hydrogen bond from VAL12 (2.33 Å), one carbon HB from VAL12 (2.32 Å), and one carbon HB from VAL13 (3.45 Å). PSM22 has more affinity to the target and a docking score is 35% higher (−4.61 kcal/mol) than YSM21, resulting from several electrostatic interactions with imidacloprid within 4 Å in length. Two conventional HBs can be noticed, including one from GLN18 (2.61 Å) and one from ALA8 (4.00 Å). Two carbon HBs from TRP15 (2.40 Å) and GLN18 (2.38 Å) are also observed with PSM22 and IMI. The binding energy of peptide PSW31 is even higher than that found with PSM22 (−4.80 kcal/mol). The receptor (PSW31) established two carbon hydrogen interactions of less than 2.70 Å with SER5 (2.66 Å) and with SER7 (2.60 Å). WQA34 has the lowest binding affinity value (highest docking score) (−5.30 kcal/mol) among YSM21, PSM22, PSW31, and WQW34. WQW34 shows one hydrogen bond interaction having a length of less than 4 Å with the target from TRP6 (3.91 Å). As seen from the data in Table 1, the docking scores of the shorter peptides are lower than those observed for the longer peptides. RNR12 is a specific oligopeptide sequences for the recognition of imidacloprid [Ding, X. et al., Oligopeptides functionalized surface plasmon resonance biosensors for detecting thiacloprid and imidacloprid, Biosens Bioelectron, 2012, 35, 1, 271-6, which is incorporated herein by reference in its entirety]. Docking scores were shown to increase after the lengthening and the combination of the 3 primary selected peptides. YSM21 has the lowest score among the four longer peptides (−4.30 kcal/mol), but it is higher than the best score found for WQW13 and the reference RNR12 peptide. Increasing the length of a sequence of the peptides increases the binding energy, which may be due to a decrease of peptide flexibility. The 3D structure of RNR12 and the interactions from molecular docking results are shown in FIGS. 8A-8B. FIG. 8B depicts RNR12 having two interactions with imidacloprid resulting from interactions with ARG3 and LEU7.

Further work has been done with YSM21, PSM22, PSW31, and WQA34, and the results were compared their results to that obtained with the reference RNR12 peptide. All the data, gathered in Table 2, indicate that the corresponding molecular systems are energetically favorable since they all show negative free energy values. RNR12 has an average free energy of −2.29±0.25 kcal/mol, which is higher than that obtained with PSW31 and PSM22. YSM21 and WQA34 resulted in average free energy values lower than that obtained with RNR12. YSM21 and WQA34 have average free energy values lower by 154.6±3.2% and 281.2±1.1% with respect to the average free energy value obtained for RNR12. These two peptides could potentially serve as a better recognition of the target molecule than the reference peptide. Furthermore, energy decomposition showed that a large contribution to the free energy arises from van der Waals interactions, electrostatic interactions, and a non-polar free energy. The polar free energy contributed unfavorably to the interaction due all the values being positive; however, these values remain small compared to the other contributions. For example, the favorable contribution is 144.6% of the interaction energy while the unfavorable contribution is 40.5%. The difference comes from two weak terms, which are a favorable entropic term and an unfavorable dispersion free energy term, neglected in Eq 2.

YSM21, PSM22, and PSW31 have high dissociation constants (1.4×10⁴ to 3.4×10⁴ μM), which are on the same order of magnitude of the reference peptide. This denotes that these complexes are not very stable because their relatively low average free binding energies. Peptide WQA34 has a dissociation constant of 40 μM, which is 3 orders of magnitude lower than the reference RNR12 peptide, making this in-silico designed peptide a viable candidate for the recognition of imidacloprid and building sensors for it.

TABLE 2
The average free energies result in kcal/mol for the solvated systems, details of
the energy contributions for the different complexes and the dissociation constant^{a}

| | ΔE_{Vdw} | ΔE_{elec} | ΔGₚₒₗ | ΔGₙₒₙₚₒₗ | ΔG_{(binding, slvd)} | |
|---|---|---|---|---|---|---|
| Complexes | (kcal/mol) | (kcal/mol) | (kcal/mol) | (kcal/mol) | (kcal/mol) | K_{d} (μM) |
| YSM21/IMI | −2.14 | −0.88 | 0.63 | −1.26 | −3.54 ± 0.08 | 1.4 × 10⁴ |
| PSM22/IMI | −1.55 | −0.60 | 0.75 | −1.14 | −2.64 ± 0.07 | 1.2 × 10⁴ |
| PSW31/IMI | −1.85 | −0.67 | 1.49 | −1.17 | −2.43 ± 0.21 | 3.4 × 10⁴ |
| WQA34/IMI | −5.30 | −1.95 | 2.61 | −2.06 | −6.44 ± 0.27 | 40 |
| RNR12/IMI | −1.37 | −0.80 | 1.05 | −1.11 | −2.29 ± 0.25 | 3.4 × 10⁴ |

^{a}The average free energy of solvated receptor-ligand binding (ΔG_{binding, slvd}) and the four main contributions from Van der Waals interactions (ΔE_{vdW}), electrostatic interactions (ΔE_{elec}), polar solvation free energy (ΔGₚₒₗ), and nonpolar solvation free energy (ΔGₙₒₙₚₒₗ). K_{d} represents an equilibrium dissociation constant

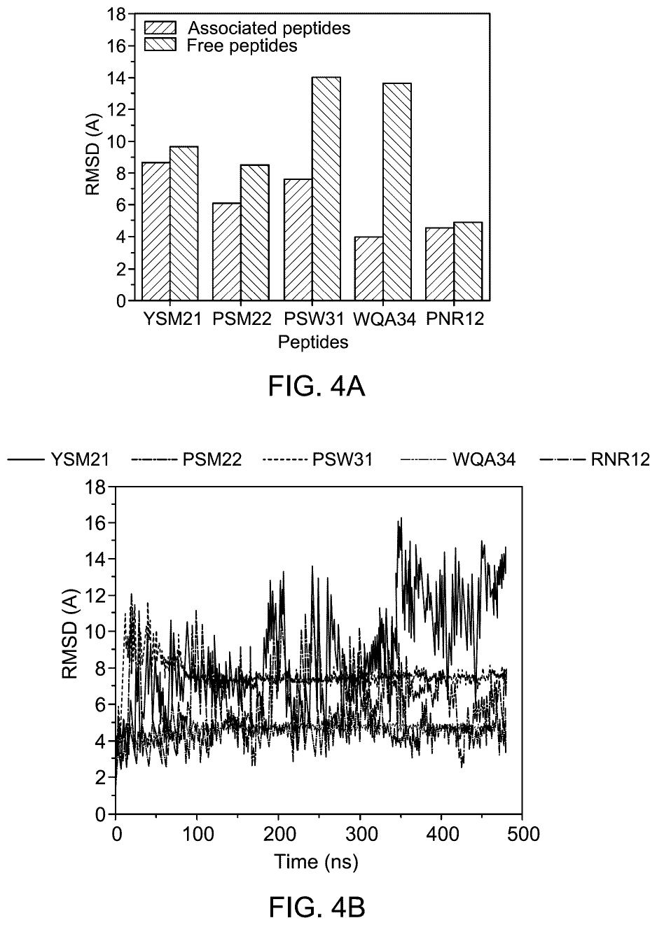

The RMSD analysis indicates that there are changes in the IMI-peptide interactions between free and associated states. Specifically, the RMSD values of PSW31 and WQA34 show a decrease of 50% and 74%, respectively, indicating a more stable interaction of the peptide with IMI in the associated state versus the peptide in a free state with no IMI, as seen in FIG. 4A. The reference RNR12 peptide displayed an associated peptide RMSD value almost superposable to that of WQA34, showing that both peptides are stable with IMI. YSM21 exhibits minor variations in RMSD values of free and associated peptides that do not exceed 10% of each other. This suggests that the peptide-IMI interaction of YSM21 is stable. From FIG. 4B, it can be observed that a large fluctuation of RMSD values occurred. This denotes that the ligand keeps changing confirmation during the overall simulation time. PSM22 displayed a decrease in the average RMSD value of 30% in the associated state compared to the free state, indicating that the complex formation has a stabilizing effect on the peptide conformation.

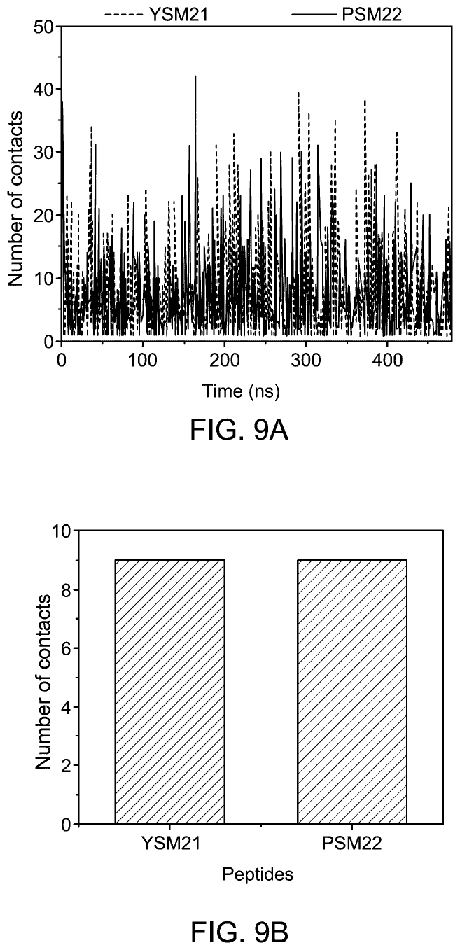

These results provide insights into IMI-peptide interactions and their dynamics, which can be useful in understanding the underlying mechanisms of biological processes. Time-dependent RMSD analysis reveal PSW31 and WQA34 reach stability after 100 ns of simulation, while YSM21 and PSM22 show RMSD fluctuations until the end of the simulation time, although they have a number of contacts (9) with the target, as shown in FIGS. 9A-9B. This difference in stability may be attributed to the length of the peptide sequences, which is increased from 31 to 34 amino acids in the case of PSW31 and WQA34. The longer peptide sequences may provide more stabilizing interactions with the pesticide molecule and reduce the fluctuation in the IMI-peptide complex structure, leading to a faster stabilization. Conversely, the shorter peptide sequences of YSM21 and PSM22 may not provide enough stabilizing interactions, resulting in higher structural fluctuations and slower stabilization. These findings demonstrate consideration of length and sequence of peptides in studying their interactions with proteins and the dynamic behavior of imidacloprid-peptide complexes over time.

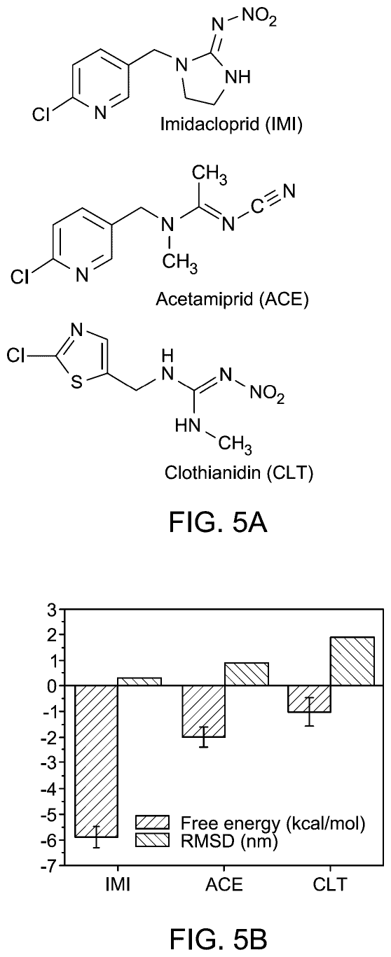

Selectivity of the lead peptide (i.e., WQA34) was examined by comparing it with two other pesticides. The first one is acetamiprid (ACE) [Hamami, M. et al., Self-Assembled MoS2/ssDNA Nanostructures for the Capacitive Aptasensing of Acetamiprid Insecticide, Applied Sciences, 2021, 11, 4, 1382, which is incorporated herein by reference in its entirety], and the second one is clothianidin (CLT) [Brown, L. A. et al., Neonicotinoid insecticides display partial and super agonist actions on native insect nicotinic acetylcholine receptors, J Neurochem, 2006, 99, 2, 608-15; and Dai, P. et al., Chronic toxicity of clothianidin, imidacloprid, chlorpyrifos, and dimethoate to Apis mellifera L. larvae reared in vitro, Pest Manag Sci, 2019, 75, 1, 29-36, which are incorporated herein by references in their entirety], which are two widely used neonicotinoid insecticides from the same family and are seen in FIG. 5A [Dai, P. et al., Chronic toxicity of clothianidin, imidacloprid, chlorpyrifos, and dimethoate to Apis mellifera L. larvae reared in vitro, Pest Manag Sci, 2019, 75, 1, 29-36; and Hamami, M. et al., Self-Assembled MoS2/ssDNA Nanostructures for the Capacitive Aptasensing of Acetamiprid Insecticide, Applied Sciences, 2021, 11, 4, 1382, which are incorporated herein by references in their entireties]. As shown in FIG. 5B, the MM-PBSA results of IMI, ACE, and CLT are −5.88±0.38 kcal/mol, −2.00±0.48 kcal/mol, and −1.02±0.55 kcal/mol, respectively, demonstrating a higher affinity of WQA34 to IMI. This is also supported by the RMSD values (IMI: 0.3, ACE: 0.9, CLT: 1.9). A higher stabilization of the complex was formed with IMI over those formed with ACE or CLT. The RSMD value of CLT is approximately six times higher than that found for IMI and its docking score is lower by 66%. This suggests that the selected peptide is potentially selective for IMI in the following order: IMI>ACE>CLT.

Starting from a nicotinic receptor-imidacloprid complex available in the PDB databank, three primary short peptides were selected that can complex the target molecule with energies close to that obtained from a reference RNR12 peptide. Combination of these peptides allow for lengthening and increased of the overall stability. The WQA34 peptide has a threefold higher affinity for imidacloprid and a dissociation constant that is 850 times lower compared to the reference RNR12 peptide. WQA34 showed a stable complex formed with imidacloprid, as demonstrated by MD simulations. Furthermore, the peptide was shown to be selective to the target pesticide (IMI) against two other members from the same family (ACE and CLT). WQA34 may be used in wet-lab experiments to selectively recognize the target pesticide.

Numerous modifications and variations of the present disclosure are possible in light of the above teachings. It is therefore to be understood that within the scope of the appended claims, the disclosure may be practiced otherwise than as specifically described herein.

1: A method of pesticide detection, comprising:
contacting at least one peptide with a sample comprising a pesticide,
wherein the peptide is selected from a group consisting of YSM21, PSM22, PSW31, and WQA34,
wherein the YSM21 peptide has an amino acid sequence at least 85% identical to SEQ. ID No.: 1,
wherein the PSM22 peptide has an amino acid sequence at least 85% identical to SEQ. ID No.: 2,
wherein the PSW31 peptide has an amino acid sequence at least 85% identical to SEQ. ID No.: 3,
wherein the WQA34 peptide has an amino acid sequence at least 85% identical to SEQ. ID No.: 4,
wherein the peptide comprises a fluorescent probe,
wherein the pesticide is imidacloprid,
binding the pesticide to the peptide to form an analyte composition; and
detecting the pesticide based on a fluorescence spectrum of the analyte composition.

2: The method of claim 1, wherein the peptide has a binding affinity for imidacloprid of −3 to −6 kcal/mol.

3: The method of claim 1, wherein the peptide is WQA34, and a hydroxyl group of the imidacloprid interacts with a tryptophan in a sixth position of the WQA34 during the binding.

4: The method of claim 1, wherein the peptide is WQA34, and a benzene ring of the imidacloprid interacts with a tryptophan in a thirty fourth position of the WQA34 during the binding.

5: The method of claim 1, wherein the peptide is WQA34, and a chloride atom of the imidacloprid interacts with a tryptophan in a thirty fourth position of the WQA34 and a proline in a fifteenth position of the WQA34 during the binding.

6: The method of claim 1, wherein the peptide is WQA34, and the peptide has an average binding free energy of −7 to −6 kcal/mol to the imidacloprid during the binding.

7: The method of claim 1, wherein the peptide is WQA34, and the peptide has an average free energy of solvated receptor-ligand binding of −6 to −4 kcal/mol to the imidacloprid during the binding.

8: The method of claim 1, wherein the peptide is WQA34, and the peptide has an equilibrium dissociation constant of 30 to 50 μM to the imidacloprid during the binding.

9: The method of claim 1, wherein the peptide is WQA34, and a root mean square deviation value of the peptide associated with the imidacloprid during the binding is 70 to 80% less compared to a root mean square value of the peptide not associated with the imidacloprid during the binding.

10: The method of claim 1, wherein the peptide is PSW31, and a root mean square deviation value of the peptide associated with the imidacloprid during the binding is 45 to 55% less compared to a root mean square value of the peptide not associated with the imidacloprid during the binding.

11: The method of claim 1, further comprising:
binding the peptide to acetamiprid and clothianidin.

12: The method of claim 11, wherein the peptide is WQA34, and the peptide has an average free energy of −3 to −1 kcal/mol for acetamiprid during the binding.

13: The method of claim 11, wherein the peptide is WQA34, and the peptide has an average free energy of −2 to 0 kcal/mol for clothianidin during the binding.

14: The method of claim 1, wherein the peptide is YSM21 and has an amino acid sequence at least 95% identical to SEQ. ID No.: 1.

15: The method of claim 1, wherein the peptide is PSM22 and has an amino acid sequence at least 95% identical to SEQ. ID No.: 2.

16: The method of claim 1, wherein the peptide is PSW31 and has an amino acid sequence at least 95% identical to SEQ. ID No.: 3.

17: The method of claim 1, wherein the peptide is WQA34 and has an amino acid sequence at least 90% identical to SEQ. ID No.: 4.

18: 18: The method of claim 1, wherein the peptide is WQA34 and has an amino acid sequence at least 95% identical to SEQ. ID No.: 4.

19: The method of claim 1, wherein the peptide is WQA34 and has an amino acid sequence at least 99% identical to SEQ. ID No.: 4.

20: The method of claim 1, wherein the peptide is PSW31, and the peptide has an equilibrium dissociation constant of 30,000 to 40,000 μM to the imidacloprid during the binding.
